(19) United States
(12) Patent Application Publication
(10) Pub. No.: US 2007/0093716 A1
(43) Pub. Date: Apr. 26, 2007

(54) Method and apparatus for elasticity imaging

(75) Inventor: Emil G. Radulescu, New Haven, CT (US)

Correspondence Address: BACHMAN & LAPOINTE, P.C., 900 CHAPEL STREET, SUITE 1201, NEW HAVEN, CT, 06510, US

(73) Assignee: ALOKA CO., LTD.

(21) Appl. No.: 11/387,635

(22) Filed: Mar. 22, 2006

Related U.S. Application Data
(60) Provisional application No. 60/730,709, filed on Oct. 26, 2005.

(51) Int. Cl. A61B 8/00

(52) U.S. Cl. 600/437

(57) ABSTRACT

A computational efficient algorithm for compression analysis of free-hand static elasticity imaging performed using medical diagnostic ultrasound imaging equipment offers tissue compression quality and quantity feedback to the operator. The algorithm includes a criterion for automatic selection of the most advantageous pre- and post-compression frame pairs delivering elasticity images of optimal dynamic ranges (DR) and signal-to-noise ratios (SNR). The use of the algorithm in real time eases operator training and reduces significantly the amount of artifact in the elasticity images while lowering the computational burden.

### CROSS-REFERENCE TO RELATED APPLICATION

Benefit is claimed of U.S. patent application Ser. No. 60/730,709, filed on Oct. 26, 2005, and entitled “Method and Apparatus for Elasticity Imaging”, the disclosure of which is incorporated by reference herein as if set forth at length.

### BACKGROUND OF THE INVENTION

1. Field of the Invention

The present invention relates to a computational efficient algorithm for tissue compression analysis for free-hand static elasticity imaging. More specifically, this invention relates to an elasticity imaging system that employs medical diagnostic ultrasound imaging equipment to produce strain images.

2. Description of Related Art

It has been proved that pathological conditions often produce changes in biological tissue stiffness. Tumor tissues, for example, are known to exhibit mechanical properties different from the surrounding tissue, as indicated by the use of palpation as a diagnostic tool. Breast and prostate tumors are especially susceptible to changes in mechanical properties, as indicated in an article by T. A. Krouskop, T. M. Wheeler, F. Kallel, B. S. Garra, and T. Hall, entitled “Elastic moduli of breast and prostate tissues under compression.”, Ultrasonic Imaging, 20:260-274, 1998, which is incorporated by reference herein.

Many cancers, such as scirrhous carcinoma of the breast, appear as extremely hard nodules. However, a lesion may or may not possess echogenic properties that would make it detectable with conventional diagnostic ultrasound imaging systems. Tumors of the prostate or the breast may thus be difficult to distinguish with conventional ultrasound techniques, yet may still be much stiffer than the surrounding tissue, as reported in an article by B. S. Garra, I Cespedes, J. Ophir, S. Spratt, R. A. Zuurbier, C. M. Magnant, and M. F. Pennanen, entitled “Elastography of breast lesions; initial clinical results,” Radiology, 202:79-86, 1997, which is incorporated by reference herein. As the echogenity and the stiffness of tissue are in general uncorrelated, Garra et al. observe it is expected that imaging the hardness of the biological tissue will provide new information related to the pathological conditions, facilitating the diagnosis process.

The experimentally obtained elastic modulus data in normal and abnormal breast tissues at different frequencies and precompression strain levels was reported in the aforementioned article “Elastic moduli of breast and prostate tissues under compression.” The data in the article shows that the differences between the elastic moduli of the various tissues of the breast may be useful in developing methods to distinguish between benign and malignant tumors. Tissues of the prostate were also examined as cancers of the prostate are also significantly stiffer than normal tissue. Similar data indicating differences between the elastic moduli for normal and abnormal prostate tissues were also reported.

The imaging modality that facilitates the display of mechanical properties of biological tissue is called elastography. The purpose of elastography is to display an image of the distribution of a physical parameter related to the mechanical properties of the tissue for clinical applications. In addition to the aforementioned breast and prostate applications of elastography, successful results have been reported for muscle and myocardial applications by F. Kallel, J. Ophir, K. Magee, and T. A. Krouskop, entitled “Elastographic imaging of low-contrast elastic modulus distributions in tissue.”, Ultrasound in Med. & Biol, 24(3): 409-425, 1998; E. E. Konofagou, J. D'Hooge, and J. Ophir, entitled “Myocardial elastography—a feasible study in vivo.”, Ultrasound in Med. & Biol. 28(4):475-482, 2002, which is incorporated by reference herein.

Elasticity imaging consists of inducing an external or internal motion to the biological tissue and evaluating the response of the tissue using conventional diagnostic ultrasound imaging and correlation techniques. Depending on the imaging mode and on the nature of tissue motion, elasticity imaging applications are divided into three distinct categories: a) static elasticity (also known as strain-based, or reconstructive) that involves imaging internal motion of biological tissue under static deformation; b) dynamic elasticity (also known as wave-based) that involves imaging shear wave propagation through the tissue; and, c) mechanical elasticity (also known as stress-based and reconstructive) that involves measuring surface stress distribution of the tissue.

Each of the three elasticity imaging applications comprises three main functional components. First, the data are captured during externally or internally applied tissue motion or deformation. Second, the tissue response is evaluated, that is, displacement, strain, and stress are determined. Lastly, the elastic modulus of the tissue is reconstructed using the theory of elasticity. The last step involves implementing the theory of elasticity into modeling and solving the inverse problem from strain and boundary conditions to elastic modulus. As the boundary conditions and the modeling of theory of elasticity are highly dependent on the structure of the biological tissue, the implementation of the last step is rather cumbersome and typically not performed. Moreover, the evaluation and display of tissue strain in the second step is considered to deliver an accurate reproduction of the tissue's mechanical properties.

Static elasticity imaging application is the most frequently used modality. In this application, a small quasi-static compressive force is applied to the tissue using the ultrasound imaging transducer. The force can be applied either using motorized compression fixtures or using freehand scanning. The RF data before and after the compression are recorded to estimate the local axial and lateral motions using correlation methods. The estimated motions along the ultrasound propagation direction represent the axial displacement map of the tissue and are used to determine the axial strain map. The strain map is then displayed as a gray scale or color-coded image and is called an elastogram.

While the majority of the elasticity imaging work has been concentrated so far on off-line processing, proof of concept and method optimization, real-time oriented applications have been only recently reported by Y. Zhu and T. J. Hall, entitled “A modified block matching method for real-time freehand strain imaging.”, Ultrasonic Imaging, 24:161-176, 2002, which is incorporated by reference herein; and by T. Shiina, M. Yamakawa, N. Nitta, E. Ueno, T. Matsumura, S. Tamano, and T. Mitake, entitled “Clinical assessment of real-time, freehand elasticity imaging system based on the combined autocorrelation method.”, 2003 IEEE Ultrasonics Symposium, pages 664-667, which is incorporated by reference herein. The need for real-time elasticity imaging applications in clinical environment is primarily of a practical nature. However, real-time elasticity imaging is indeed needed to acquire and process the ultrasonic echo data in such a way that patient-scanning time is relatively low and diagnostically relevant elasticity images are produced immediately during the scan. Thus, such real-time elasticity imaging systems are capable of displaying ultrasonic B-mode images and strain images on the same screen in real-time. Such a display also facilitates the assessment of the clinical relevance of the strain images being obtained.

Furthermore, the real-time processing of the ultrasonic echo data allows for freehand compression and scanning of the biological tissue rather than utilizing bulky and slow motorized compression fixtures. Freehand compression, as opposed to motorized compression facilitates a more manageable and user-friendly scanning process and allows for a larger variety of scanning locations. Its disadvantage, however, consists of exhaustive operator training, as the sonographer constantly needs to adjust the compression technique to obtain strain images of good quality. In more detail, to obtain strain images of consistent dynamic range (“DR”) and signal-to-noise ratio (“SNR”), the sonographer needs to maintain a constant compression rate while avoiding lateral and out-of-plane tissue motions. Moreover, the compression has to be performed exclusively on the axial direction of the imaging transducer while maintaining a certain speed and repetition period.

In short, due to the extremely complex nature of the tissue compression, obtaining elasticity images of consistent quality using free-hand strain imaging is neither trivial nor as expeditious as obtaining good quality B-mode images, thus real-time compression feedback is necessary to ensure proper operator training.

In an attempt to overcome the limitations discussed above, a few research groups proposed and implemented real-time static elasticity imaging systems as reported by Y. Zhu and T. J. Hall, entitled “A modified block matching method for real-time freehand strain imaging.”, Ultrasonic Imaging, 24:161-176, 2002, which is incorporated by reference herein; and, by T. Shiina, M. Yamakawa, N. Nitta, E. Ueno, T. Matsumura, S. Tamano, and T. Mitake, entitled “Clinical assessment of real-time, freehand elasticity imaging system based on the combined autocorrelation method.”, 2003 IEEE Ultrasonics Symposium, pages 664-667, which is incorporated by reference herein. In addition, U.S. Pat. No. 6,508,768 B1 to Hall et al. (“'768 patent”) describes in detail a real-time static elasticity imaging procedure and implementation. However, those implementations disclosed by the '768 patent and the Zhu et al. and Shiina et al. articles do not account completely for all the limitations mentioned above.

More particularly, neither the articles by Zhu et al. and Shiina et al. nor the teachings of the '768 patent provide a quantitative indication of the compression quality being achieved by the operator. Moreover, the operator does not receive guidance in order to improve the compression quality when s/he is only provided strain images that may contain artifacts and poor SNR. One of several drawbacks being that possible artifacts present in the strain image cannot be qualitatively linked to poor compression quality. Additionally, the current implementations calculate and display strain images continuously, independently of the quality of the compression, or even in the absence of compression. Therefore the computational burden placed upon the imaging system is extremely high while only select sets of strain images faithfully indicate the mechanical properties of the imaged tissue and are artifact-free. Moreover, depending on the applied compression rate, strain images are displayed with variable (and less than optimal) DR and SNR, allowing for artifacts.

There exists a need for a computational efficient algorithm capable of providing real-time tissue compression quality and quantity feedback to the operator.

There also exists a need for a computational efficient algorithm that automatically selects the most advantageous pre- and post-compression frame pairs for delivering elasticity images of optimal dynamic ranges and signal-to-noise ratios.

There further exists a need for a computational efficient algorithm that generates compression quality feedback independently of the quality of the compression being achieved.

There exists still yet a need for a computational efficient algorithm that measures, analyzes and visually displays both the axial and lateral displacements (negative and positive) of the decompression of tissue.

There exists further still a need for a computational efficient algorithm that captures and archives all information utilized in generating the elasticity images for off-line analysis.

### SUMMARY OF THE INVENTION

In accordance with an aspect of the present invention, a process for performing elasticity imaging on a biological tissue broadly comprises selecting automatically based upon at least one criterion at least one frame pair comprising a pre-compression frame and a post-compression frame; analyzing the at least one frame pair; calculating an elasticity image; and displaying the elasticity image. The automatic selection step broadly comprises using a compression feedback algorithm. The at least one criterion broadly comprises an amount of tissue displacement and at least one tissue correlation result. The automatic selection step further broadly comprises predicting an elasticity image quality prior to calculating an elasticity image. The automatic selection step further broadly comprises providing to an operator at least one of the following: a visual feedback or an audible feedback or both visual feedback and audible feedback. The providing step further broadly comprises providing the visual feedback and the audible feedback to the operator upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion. The process also broadly comprises confirming off-line the quality of a plurality of data used in the calculation of the elasticity image. The confirmation step broadly comprises displaying visually and projecting audibly at least one of the following: at least one quantitative data, at least one qualitative data, or both at least one quantitative data and at least one qualitative data. The confirmation step also broadly comprises displaying visually or projecting audibly at least one of the following: at least one quantitative data, at least one qualitative data, or both at least one quantitative data and at least one qualitative data.

In accordance with yet another aspect of the present invention, a process for performing elasticity imaging broadly comprises setting a region of interest about an image; deforming a biological tissue to create a tissue deformation; acquiring at least two RF frame data at an imaging-relevant frame rate; introducing the at least two RF frame data into a compression feedback algorithm; determining at least one quantitative indication of a tissue deformation quality for the at least two RF frame data within at least one block from the region of interest using a block matching algorithm; comparing the at least one quantitative indication of the at least two RF frame data to at least one of a plurality of threshold values within at least one block from the region of interest; displaying the comparison of the at least one quantitative indication of the at least two RF frame data to at least one of the plurality of threshold values; predicting an acceptable tissue deformation based upon the comparison; determining the predicted acceptable tissue deformation is satisfactory to yield a satisfactory tissue deformation; and displaying an elasticity image of the biological tissue.

In accordance with yet another aspect of the present invention, an ultrasound system broadly comprises a computer readable storage device readable by the system, tangibly embodying a program having a set of instructions executable by the system to perform the following steps for performing elasticity imaging, the set of instructions broadly comprise an instruction to set a region of interest about an image followed by the deformation of a biological tissue to create a tissue deformation; an instruction to acquire at least two RF frame data at an imaging-relevant frame rate; an instruction to introduce the at least two RF frame data into a compression feedback algorithm; an instruction to determine at least one quantitative indication of a tissue deformation quality for the at least two RF frame data within at least one block from the region of interest using a block matching algorithm; an instruction to compare the at least one quantitative indication of the at least two RF frame data to at least one of a plurality of threshold values within at least one block from the region of interest; an instruction to display the comparison of the at least one quantitative indication of the at least two RF frame data to at least one of the plurality of threshold values; an instruction to predict an acceptable tissue deformation based upon the comparison; an instruction to determine the predicted acceptable tissue deformation is satisfactory to yield a satisfactory tissue deformation; and an instruction to display an elasticity image of the biological tissue.

An ultrasound system comprising a computer readable storage device readable by the system, tangibly embodying a program having a set of instructions executable by the system to perform the following steps for performing elasticity imaging, the set of instructions broadly comprises an instruction to select automatically based upon at least one criterion at least one frame pair comprising a pre-compression frame and a post-compression frame; an instruction to analyze the at least one frame pair; an instruction to calculate an elasticity image; and an instruction to display the elasticity image. The automatic selection instruction broadly comprises an instruction to use a compression feedback algorithm. The at least one criterion broadly comprises an amount of tissue displacement and at least one tissue correlation result. The automatic selection instruction further broadly comprises an instruction to predict an elasticity image quality prior to calculating an elasticity image. The automatic selection instruction also further broadly comprises an instruction to provide to an operator at least one of the following: a visual feedback or an audible feedback or both said visual feedback and said audible feedback. The providing instruction further broadly comprises an instruction to provide the visual feedback and the audible feedback to the operator upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion. The ultrasound system further broadly comprises an instruction to confirm off-line the quality of a plurality of data used in the calculation of the elasticity image. The confirmation instruction broadly comprises an instruction to display visually and project audibly at least one of the following: at least one quantitative data, at least one qualitative data, or both at least one quantitative data and at least one qualitative data. The confirmation instruction broadly comprises an instruction to display visually or project audibly at least one of the following: at least one quantitative data, at least one qualitative data, or both at least one quantitative data and at least one qualitative data.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a block diagram of a real-time, free-hand static elasticity imaging system utilizing a diagnostic ultrasound system, incorporating a compression feedback algorithm of the present invention;

FIG. 2 is a flowchart illustrating the main components and functionality of a compression feedback algorithm;

FIG. 3 is a diagram of a B-Mode image display of an RF reference frame buffer, the elasticity imaging region of interest before compression and a region of interest after compression;

FIG. 4 is a graph showing the cumulated axial displacement of an elasticity imaging region of interest reference points for different depths along the acoustic axis;

FIG. 5 is a color coded diagram showing the cumulated lateral displacement of an elasticity imaging region of interest reference points for different depths along the acoustic axis;

FIG. 6 is a chart showing the average quantitative indication of tissue compression quality for different depths;

FIG. 7 is a graph depicting unacceptable compression as the axial displacement of one of the elasticity imaging reference points is greater than a predefined maximum acceptable axial threshold;

FIG. 8 is a graph depicting unacceptable compression as the axial displacement of several of the elasticity imaging reference points possess negative values; and

FIG. 9 is a graph depicting acceptable compression yet failing to produce good quality strain images due to axial displacements smaller than an imaging acceptable threshold.

### DETAILED DESCRIPTION OF THE INVENTION

An elasticity imaging system, and method for using same, employs a tissue compression analysis algorithm for free-hand static elasticity imaging utilizing medical diagnostic ultrasound imaging equipment. The compression feedback algorithm's application offers tissue compression quality and provides quantity feedback to the operator. The compression feedback algorithm analyzes the pre- and post-compression frame pairs and provides an elasticity image quality prediction before an elasticity imaging module computes the elasticity image. The algorithm includes a criterion for the automatic selection of the most advantageous pre- and post-compression frame pairs for delivering elasticity images of optimal dynamic ranges and signal-to-noise ratios. The use of the algorithm in real time eases operator training and reduces significantly the amount of artifact in the elasticity images while also lowering the computational burden. In addition, operator training and confirmation of the quality of data behind the elasticity imaging results may be evaluated by displaying visually, alone or in combination, any and/or all of the qualitative, quantitative, and the like, data utilized in generating the elasticity images.

The algorithm initially considers the first frame of RF data received as the reference frame. The algorithm may then compare consecutive RF data frames using a block-matching process step. The block matching process step generally comprises applying an array measuring X number of rows and Y number of columns, where both X and Y may be, but are not limited to, odd numerals. To speed up the execution, this comparison may be executed utilizing a limited number of searching blocks.

In a preferred embodiment, the block matching algorithm may be implemented using, for example, a normalized correlation technique, a non-normalized correlation technique, and preferably a correlation coefficient technique. For each block, the search zone is limited to a small section of the following frame of RF data to speed up the execution. The search may be performed both axially and laterally. The motion of the blocks detected between consecutive frames may be given by the displacements corresponding to the lags that exhibit a maximum envelope of the correlation coefficient. The displacements found are cumulated from one frame pair to the next one.

The quantitative indication of the tissue compression quality may be given for each block by the correlation between the envelope of the reference frame and the envelope of the most current frame. A quantitative indication may be obtained by employing normalized correlation techniques and compensating for tissue motion using the displacements previously cumulated from one frame pair to the next frame pair. For display and comparison with threshold values, the quantitative data corresponding to the blocks positioned at the same depth in the ROI may be processed using a suitable technique known to one of ordinary skill in the art and displayed for each individual depth considered. Preferably, the quantitative data may be presented for three depths, which corresponds to a top line, a middle line and a bottom line of the ROI.

The compression corresponding to a given RF frame data is accepted as valid once the quantitative indication exceeds a certain threshold, the absolute value of the cumulated lateral displacement is smaller than a given threshold and the cumulated axial displacement is positive and smaller than a given threshold. Thus, a positive axial displacement indicates a compression motion rather than a decompression motion.

For a predicted acceptable tissue compression , if the cumulated axial displacement is larger than a preset imaging threshold, an originally stored RF reference frame and a given RF frame are sent to the static elasticity imaging module. The module calculates and displays a strain image in parallel with a B-Mode image of the RF reference frame. Then, the given RF frame is stored as a reference frame, the cumulated axial and lateral displacements are reinitialized and the algorithm restarts. If, however, the cumulated axial displacement is not larger than the preset imaging threshold, the compression feedback algorithm predicts the tissue compression is not large enough. The algorithm is then repeated for the next RF frame data cumulating the new displacements to the previously calculated ones.

On the other hand, if the predicted tissue compression is not acceptable, the given RF frame is stored as a reference, the cumulated axial and lateral displacements are reinitialized and the algorithm restarts without initiating a strain image display. The choice of the quantitative indication, lateral, and axial thresholds depends upon the B-Mode imaging parameters and the settings of the static elasticity imaging module.

As will be discussed in greater detail, an acceptable tissue compression, or an acceptable tissue decompression, may be quantitatively displayed as a set of points located within a range of acceptable axial threshold values. A tissue compression motion may include a set of points indicating positive axial compression values. For a compression motion, a range may generally comprise a lower threshold boundary representing a minimum axial threshold value or imaging acceptable threshold value at which an acceptable strain image may be generated, and an upper axial threshold boundary representing a maximum threshold value or a largest acceptable axial threshold value at which an acceptable strain image may be generated. In contrast, a tissue decompression may include a set of points indicating negative axial compression values. For decompression motion, a range for generating an acceptable strain image may generally comprise a lower axial threshold boundary representing a largest acceptable axial displacement absolute value, and an upper axial threshold boundary representing a minimum axial displacement absolute value or an imaging acceptable threshold value.

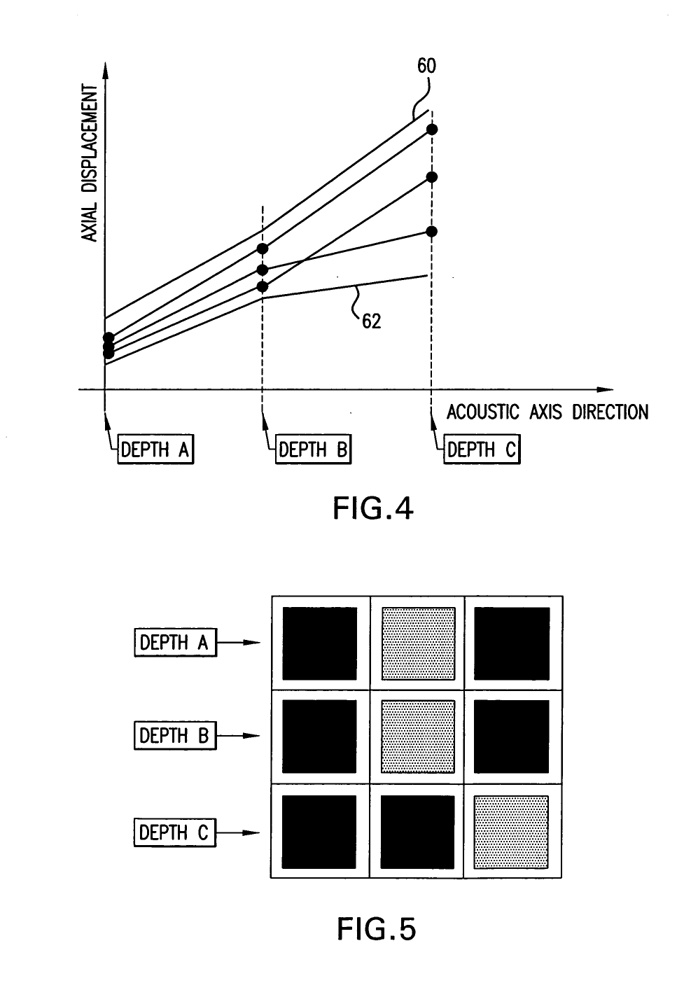

A set of points comprising an acceptable compression, or an acceptable decompression, may be displayed across either an axial displacement, as exemplified above, or a lateral displacement, respectively. Likewise, a range of acceptable threshold values may also be displayed across either the axial displacement or the lateral displacement, respectively. Such a quantitative display may be generated for both positive compression values (compression motions) and negative decompression values (decompression motions). For example, FIGS. 4 through 8 illustrate quantitative displays of both acceptable and unacceptable compressions using positive compression values across an axial displacement.

The present invention, while herein described with respect to real-time, free-hand static elasticity imaging, is not so limited. Rather, a compression feedback algorithm may also be implemented in a static elasticity imaging system using motorized compression fixtures and off-line data processing. Additionally, with appropriate modifications contemplated herein, a compression feedback algorithm may also be implemented in a dynamic elasticity imaging system.

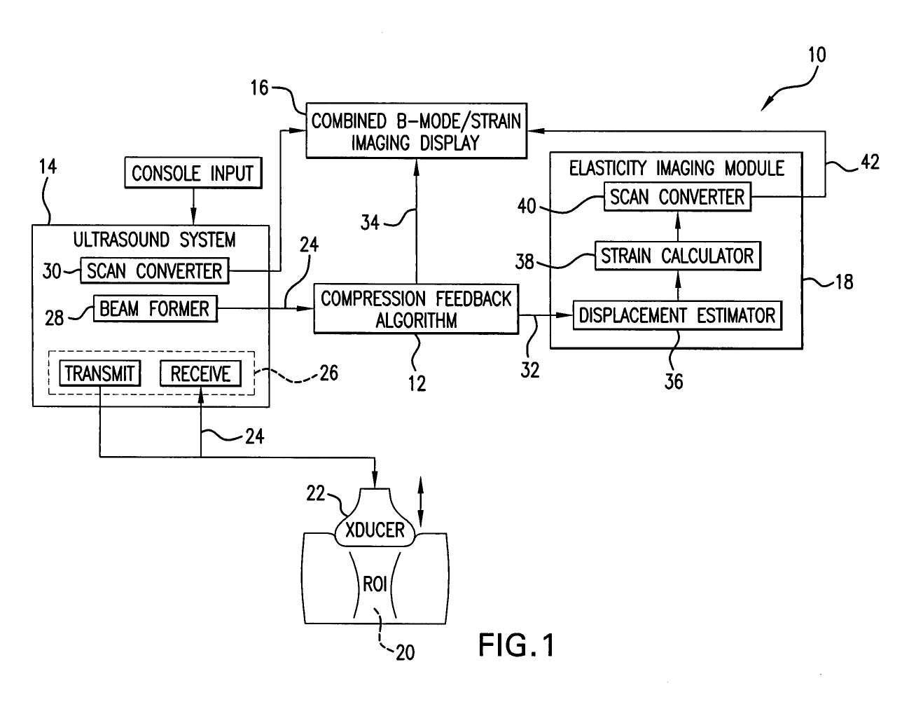

Referring generally to FIGS. 1-8, in free-hand, real-time, static elasticity, the operator sets a region of interest (hereinafter “ROI”) within a B-Mode image obtained from an ultrasound diagnostic system and compresses cyclically a biological tissue under investigation using, for example, an ultrasonic transducer probe. The ultrasound system acquires RF data in real-time, that is, at imaging-relevant frame rates, and sends it to the compression feedback algorithm.

Referring now to FIG. 1, the algorithm may be integrated in a static, free-hand, real-time elasticity imaging system 10. Elasticity imaging system 10 includes, in addition to compression feedback algorithm 12, the aforementioned diagnostic ultrasound system 14, a combined B-Mode/strain imaging display unit 16 and an elasticity imaging module 18.

In free-hand, real-time, static elasticity, the operator sets a region of interest (“ROI”) 20 within a B-Mode image obtained from ultrasound diagnostic system 14. The ROI may be set about a part of an image such that the RF data is limited, or may be set about the entire image and constitutes the entire image. The operator may deform, for example, compress, decompress or twist, the tissue under investigation within the ROI using ultrasonic transducer probe 22. Ultrasound system 14 acquires RF frame data 24 at imaging-relevant frame rates, that is, in real-time. The RF frame data 24 generally consists of at least two data frames in sequence. Once the RF frame data 24 is acquired, ultrasound system 14 sends RF frame data 24 to compression feedback algorithm 12.

Diagnostic ultrasound system 14 may include a console input (not shown), a transmit/receive hardware 26, as well as a beamformer module 28 and a scan converter module 30. The B-Mode images produced by scan converter 30 are sent to combined B-Mode/strain imaging display unit 16. Beamformer module 28 provides RF data in a continuous mode to compression feedback algorithm 12. Depending upon the compression quality and quantity, compression feedback algorithm 12 initiates an elasticity image by forwarding a select pair of RF data frames 32 to the elasticity imaging module 18. For each RF frame received, compression feedback algorithm 12 makes a sum of compression analysis parameters 34 available to combined B-Mode/strain imaging display 16.

Elasticity imaging module 18 may include a displacement estimator algorithm 36, a strain calculator module 38 and a scan converter 40. Displacement estimator module 36 assesses the tissue motion between RF data frames 32 received from the compression feedback algorithm 12. Strain calculator module 38 calculates the spatial derivative of the axial displacements and that result is transformed into a strain image 42 by elasticity imaging scan converter module 40. Finally, strain image 42 is sent to combined B-Mode/strain imaging display unit 16 that displays strain image 42 on a screen together with its corresponding B-Mode image.

Generally, the compression feedback algorithm 12 selects the most advantageous pre- and post-compression frame pairs for delivering elasticity images of optimal dynamic ranges and signal-to-noise ratios. As tissue density varies, the compression feedback algorithm 12 may include additional parameters to recognize such variations in tissue density.

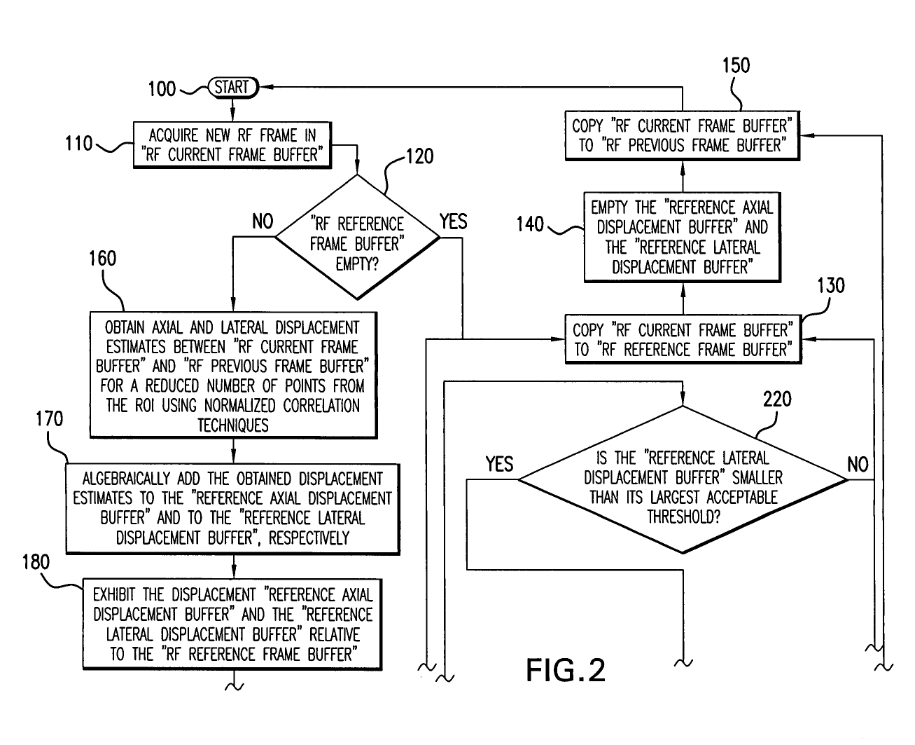

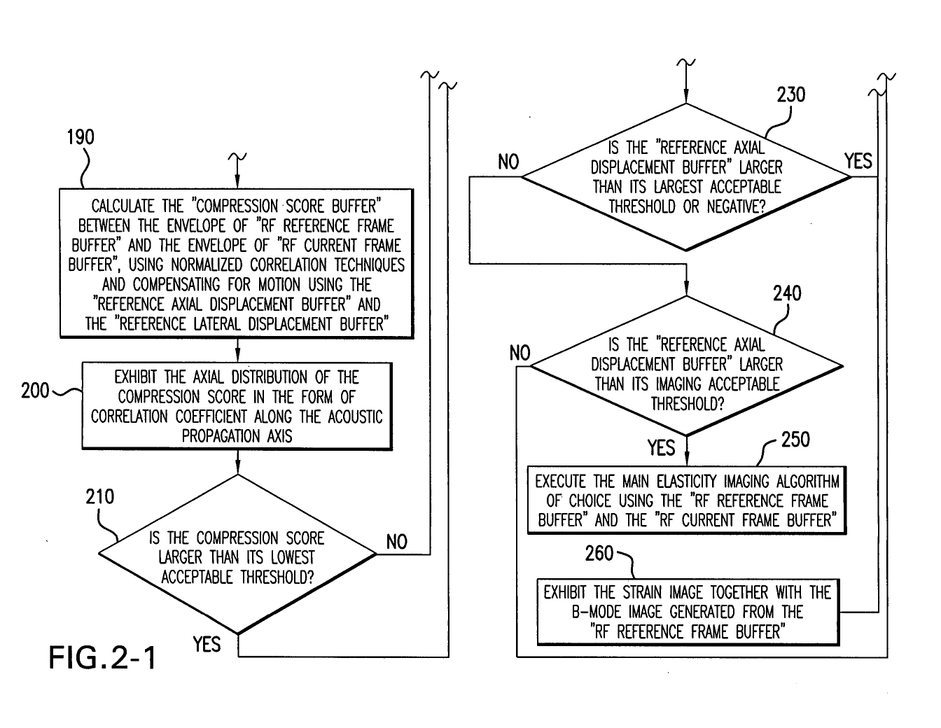

Referring now to FIG. 2, compression feedback algorithm 12 is illustrated as a flowchart. As shown, compression feedback algorithm 12 may include, but is not limited to, a plurality of buffers, each holding key data needed to perform the outlined functionality. Table 1 generally describes the buffers, their respective functionalities and relations to one another within the execution of algorithm 12.

TABLE 1

| Buffer name | Buffer description |
|---|---|
| RF Current Frame | Buffer where the current RF frame data are |
| | stored. This buffer receives new data every |
| | time the algorithm restarts, independently on |
| | the quality of the compression. |
| RF Previous Frame | Buffer that contains the RF frame data |
| | acquired one step before the data from the RF |
| | Current Frame Buffer. This buffer receives |
| | new data every time the algorithm restarts, |
| | independently on the quality of the |
| | compression. |
| RF Reference Frame | Buffer that contains the reference RF frame |
| | data. This buffer receives new data when the |
| | algorithm runs for the first time, when the |
| | compression is considered unsatisfactory or |
| | after the execution of the elasticity imaging |
| | algorithm. |
| Reference Axial | Buffer that stores the cumulated axial tissue |
| Displacement Buffer | displacements detected between the data from |
| | the RF Current Frame Buffer and the RF |
| | Reference Frame Buffer. |
| Reference Lateral | Buffer that stores the cumulated lateral |
| Displacement Buffer | tissue displacements detected between the data |
| | from the RF Current Frame Buffer and the RF |
| | Reference Frame Buffer. |
| Compression Score | Buffer that stores the compression |
| Buffer | quantitative score between the envelope of the |
| | data from the RF Current Frame Buffer and the |
| | envelope of the data from the RF Reference |
| | Frame Buffer. |

A starting point 100 of the flowchart of FIG. 2 indicates the acquisition of a new RF data frame 24 and storing the frame in the RF current frame buffer at a step 110. As shown in Table 1, RF current frame buffer may store the current, or the most recent, RF frame data 24 acquired, and preferably always stores the current RF frame data 24 acquired. The RF current frame buffer receives new data every time compression feedback algorithm 12 restarts, independently of the quality of the compression.

Next, if the RF reference frame buffer is empty at a step 120, the data from the RF current frame buffer is copied into it at a step 130 and algorithm 12 initializes its buffers at a step 140 and a step 150 and restarts with the acquisition of new RF frame data 24 at steps 100, 110. The existence of the reference frame is therefore assured and algorithm 12 is initialized using the first frame of RF data received as the reference frame. A reference axial displacement buffer and a reference lateral displacement buffer, which are initialized to zero if the RF reference frame buffer is empty, store the cumulated axial and lateral displacements, respectively, as indicated in Table 1. These buffers correspond to the displacements detected between the data from RF current frame buffer and RF reference frame buffer. RF previous frame buffer may also be initialized with the data from RF current frame buffer during this process. The RF previous frame buffer may contain, and preferably always contains, RF frame data 24 acquired one step before (see Table 1). Similarly with RF current frame buffer, RF previous frame buffer receives new data every time algorithm 12 restarts, independently of the quality of the compression.

As compression feedback algorithm 12 restarts and RF reference frame buffer is not found empty, consecutive data frames may be compared using a block-matching algorithm (see FIG. 2.) The comparison is carried out between the data sets from RF previous frame buffer and RF current frame buffer and may be performed using only a limited number of searching blocks. For example, the block matching array may comprise a 3×3, 3×5, 5×3, 5×5, 3×7, 7×3, 7×5, 7×7, and the like, array of nine (9), fifteen (15), twenty-one (21), twenty-five (25), thirty-five (35), forty-nine (49), and the like, searching blocks. Preferably, the block-matching process step is performed using a 3×3 array placed over the center of the ROI such that the center search block of the array overlaps the center of the ROI.

In a preferred embodiment, the block-matching algorithm may be implemented using a non-normalized correlation technique or a normalized correlation technique, for example, a correlation coefficient technique, as known to one of ordinary skill in the art. For each block, the search zone may be limited to a small section of the following frame of RF data to speed up the execution. The search may be performed both axially and laterally for a reduced number of points from the ROI at a step 160. Preferably, the search zone should be large enough to encompass the range of both axial and lateral displacements encountered between consecutive frames of RF data, for example, the RF current frame buffer and the RF previous frame buffer. By performing the search between consecutive RF data frames, rather than between the reference RF frame and the current RF frame, the search zone may be diminished significantly, thus increasing the algorithm computation speed. Additionally, the decorrelation between adjacent RF data frames is much lower than between the reference RF frame and the current RF frame. The motion of the blocks detected between consecutive frames is given by the displacements corresponding to the lags that exhibit a maximum envelope of the correlation coefficient as known by one of ordinary skill in the art. The envelope of the correlation coefficient represents the envelope function of the correlation coefficient results obtained for all the search positions from the search zone. Calculating the envelope assures only positive values and eliminates fluctuations in the correlation coefficient results. The displacements found are cumulated from one RF data frame pair to the next one. Specifically, reference axial displacement buffer for the axial displacements and reference lateral displacement buffer for the lateral displacements are updated at a step 170. Next, the updated values from reference axial displacement buffer and reference lateral displacement buffer may be sent to combined B-mode/strain imaging display module 16 at a step 180.

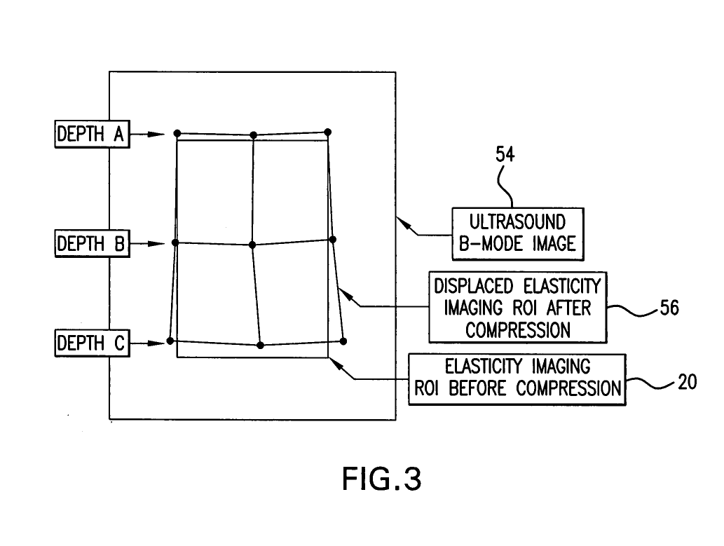

Referring now to FIG. 3, FIG. 3 illustrates a preferred embodiment of a combined B-mode/strain imaging display 16 of elasticity imaging system 10. The positions of the reference axial displacement buffer and the reference lateral displacement buffer may be superimposed onto B-mode image 54 created from RF frame data 24 contained in RF reference frame buffer. As an alternative, the scan-converted B-Mode image produced by the Scan Converter 30 can be utilized instead. The selected elasticity imaging ROI before compression 20 may be superimposed as a transparent, substantially rectangular shape onto B-mode image 54. The points for which the search is performed are displayed at the coordinates corresponding to the axial and lateral shifts contained in the reference axial displacement buffer and the reference lateral displacement buffer, respectively. For the purpose of example, and not to be considered limiting, the points may be connected by twelve (12) lines, along the horizontal and vertical axes, which indicate a displaced elasticity imaging ROI after compression 56. The image shown in FIG. 3 gives the absolute coordinates of displaced ROI 20 and offers a visual indication of how large and in what direction the compression occurred. However, the axial and lateral displacements of the ROI 56 may be significantly smaller than the size of displaced ROI 20 and, thus, unapparent to the operator. This is why the reference axial displacement buffer and the reference lateral displacement buffer may also be displayed alone on combined B-mode/strain imaging display module 16.

Referring now to FIG. 4, FIG. 4 shows the preferred display of the reference axial displacement buffer. The horizontal axis represents the depth, and “Depth A”, “Depth B” and “Depth C” corresponds to the depths marked on the vertical axis in FIG. 3. In FIG. 4 the azimuth direction is collapsed so that the points positioned at the same depth are displayed next to each other. The chart also shows a maximum acceptable axial threshold 60 and a lowest imaging acceptable threshold 62 for the reference axial displacement buffer, which will be further discussed.

Similar to the display of reference axial displacement buffer in FIG. 4, the reference lateral displacement buffer may also be shown by collapsing the azimuth direction as is understood by one of ordinary skill in the art. In another example, FIG. 5 represents another quantitative representation of the ROI. FIG. 5 shows a diagram containing nine squares that correspond to the elasticity imaging ROI reference points for different depths, for example, Depth A, Depth B and Depth C, along the acoustic axis. The absolute values of the cumulated lateral displacements exhibited in FIG. 5 are gray-coded from the color black, which indicates no displacement, to the color white, which indicates a maximum acceptable lateral displacement.

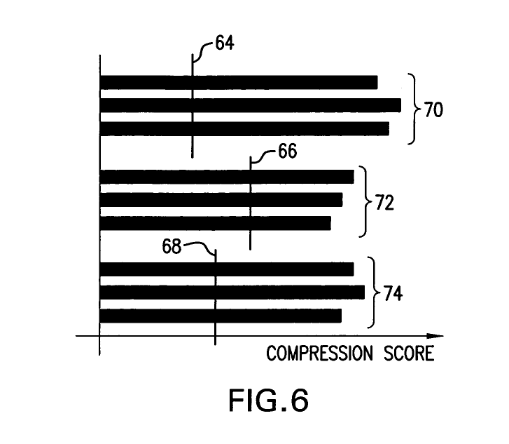

The quantitative indication of the tissue compression quality is stored in the Compression Score Buffer (see Table 1) and may be given for each block by the correlation between the envelope of the reference frame and the envelope of the most current frame. The quantitative indication may be obtained by employing normalized correlation techniques and compensating for tissue motion using the displacements previously cumulated from one frame pair to the next frame pair. For display and comparison with threshold values, the quantitative data corresponding to the blocks positioned at the same depth in the ROI may be processed using a suitable technique known to one of ordinary skill in the art and displayed for each individual depth considered. Preferably, the quantitative data may be presented for three depths, which corresponds to a top line, a middle line and a bottom line of the ROI, as illustrated in FIG. 6.

Referring now to both FIGS. 3 and 6, in a preferred embodiment, the quantitative data may be presented for three depths corresponding to a top line (“Depth A”), a middle line (“Depth B”) and a bottom line (“Depth C”) of the ROI. The information displayed in FIGS. 3 and 6 is updated in real-time as new RF data frames 24 are acquired and made available to the compression feedback algorithm 12. Referring specifically now to FIG. 6, the compression score lower threshold boundary may accept different values for each depth position (or axial position) and lateral position to better accommodate various tissue structures. In addition, the display of at least one threshold 64, 66 and 68 for each depth A, B, C, or axial position may be provided, as shown in FIG. 6. The compression score individual values for each of the individual searching blocks at a depth A 70, a depth B 72 and a depth C 74 may be exhibited on the display 16, as illustrated in FIG. 6. Therefore, the information displayed provides real-time tissue compression quality and quantity feedback to the operator, and, additionally, the displayed information allows automatic selection of the most advantageous pre- and post-compression frame pairs. The automatic selection of the frame pairs lowers the computational burden as only selected frames are used for strain imaging calculations. The real-time display and automatic selection eases operator training and lowers the strain imaging computational burden.

Referring back to FIG. 2, a first automatic decision made with respect to the real-time tissue compression quality based upon quantitative data may be calculated using the records from the compression score buffer at a step 210 (see Table 1). Specifically, if the compression score in its unmodified form or after suitable processing known to one ordinary skilled in the art, at any depth, is lower than a compression score lowest acceptable threshold set for the given depth at step 210, the compression may be considered unacceptable and compression feedback algorithm 12 reinitializes the buffers and restarts with the acquisition to new RF frame data 24 at steps 130, 140, 150 and 100. For a given depth, the lowest acceptable threshold value of the compression score may be, on one hand, large enough to exclude one or more compression-based artifacts from the strain image(s) while, on the other hand, small enough to ensure an acceptable flux of strain images produced.

A second automatic decision based on quantitative data uses the reference lateral displacement buffer. At a step 220, if the absolute value of the lateral displacement of any of the points for which the search is performed is larger than a predefined maximum acceptable lateral threshold, the compression may be considered unacceptable and compression feedback algorithm 12 may reinitialize the buffers and restart with the acquisition of new RF frame data 24 at steps 130, 140, 150 and 100, respectively. A maximum acceptable lateral threshold value should be, on one hand, small enough to exclude the compression-based artifacts from the strain image(s) while, on the other hand, large enough to ensure an acceptable flux of strain images produced.

A third automatic decision based on quantitative data uses the Reference axial displacement buffer at a step 230. If the value of the axial displacement of any of the points for which the search is performed is larger than a predefined maximum acceptable axial threshold, or negative, the compression may be considered unacceptable and the algorithm may reinitialize the buffers and restart with the acquisition of new RF frame data 24. Only positive axial displacements are accepted as they indicate compression motions, rather than decompression motions. In the alternative, negative axial displacements may be accepted so as to indicate decompression motions, rather than compression motions. Such an alternative embodiment may be employed to educate the operator, and/or generate a more complete elasticity imaging analysis of the tissue. Strain images could then be generated during decompression as well by measuring decompression motions against a negative imaging acceptable threshold and a negative maximum acceptable axial threshold.

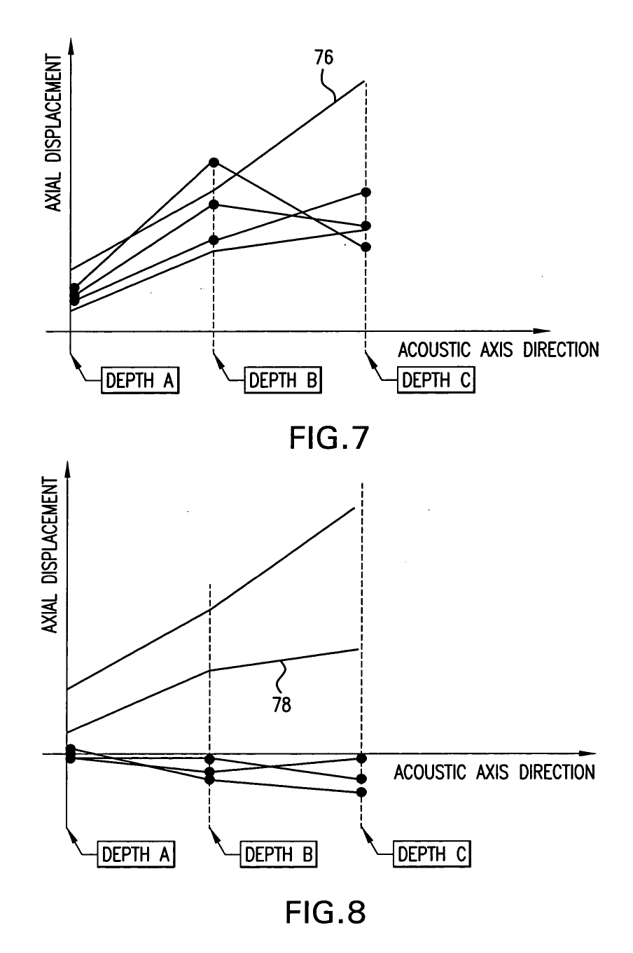

Referring now to FIG. 7, FIG. 7 illustrates an example when the value of the axial displacement of one of the points for which the search is performed is larger than a predefined maximum acceptable axial threshold 76, for example, Depth B, thus the predicted tissue compression is considered unacceptable. Similarly, FIG. 8 demonstrates another example when some of the axial displacements of the points for which the search is performed are negative and the predicted tissue compression is again considered unacceptable.

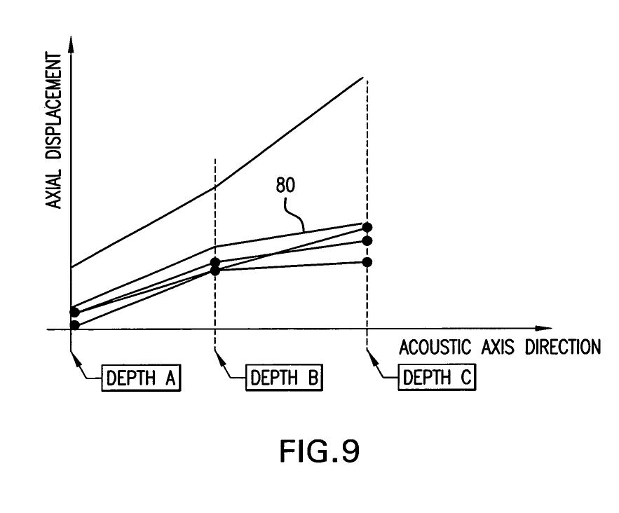

Referring again to FIG. 2, a fourth automatic decision based on quantitative data may also use Reference axial displacement buffer at a step 240. If the value of the axial displacement of any of the points for which the search is performed is smaller than a predefined imaging acceptable threshold 80, the predicted compression quality may be considered acceptable but not large enough to produce good quality strain images as is illustrated in FIG. 9. In that event, the compression feedback algorithm may restart with the acquisition of new RF frame data 24 without reinitializing the buffers.

As further illustrated in FIG. 2 at a step 250 and at a step 260, if the axial displacement of all the points for which the search is performed fall between a predefined imaging acceptable threshold and a predefined maximum acceptable axial threshold, a satisfactory tissue compression is predicted and the strain image may be calculated and displayed on combined B-Mode/strain imaging display unit 16 as demonstrated in FIG. 4. Subsequent to the strain imaging display, compression feedback algorithm 12 reinitializes the buffers and restarts with the acquisition of new RF frame data 24.

It should be noted that the positions of these thresholds with respect to depth, for example, Depth A, Depth B and Depth C, may establish the range of tissue strain at which the elasticity imaging is performed. The elasticity SNR typically exhibits a bandpass filter behavior in the strain domain as explained by T. Varghese and J. Ophir, “A theoretical framework for performance characterization of elastography: the strain filter.”, IEEE Transactions on UFFC, 44(1):164-172, 1997, which is incorporated herein by reference; and, by S. Srinivasan, R. Righetti and J. Ophir, “Trade-offs between the axial resolution and the signal-to-noise ratio in elastography.”, Ultrasound in Med. & Biol, 29(6):847-966, 2003, which is incorporated herein by reference. Therefore, the proper choice of a tissue strain range ensures an adequate elasticity signal-to-noise ratio (SNR) and, thus, an optimal elasticity dynamic range (DR).

The strain imaging DR may be optimized by appropriately setting the predefined imaging acceptable threshold near a beginning of a passband region of the strain filter and also setting a predefined maximum acceptable axial threshold close to an end of the passband region of the strain filter. The selection of strain images, and elasticity images, appearing on a display of the elasticity imaging system will be optimized for elasticity SNR and optimal elasticity DR. Compression feedback algorithm 12 may act as a filter to determine and select such strain images for display using the elasticity imaging system. Such strain images may not only enhance the quality of the results obtained by an operator, but may also enhance the operator's training.

As mentioned earlier, operator training and confirmation of the quality of data behind the elasticity imaging results may be evaluated based on the feedback provided by the elasticity imaging system. Operator training may be accomplished using one or more different methods, including but not limited to, those discussed and contemplated herein.

For example, upon completion of generating an acceptable elasticity image by the elasticity imaging module, the operator can receive feedback with respect to the quality of his/her compressions and/or decompressions in generating the elasticity image. The statistical, qualitative, quantitative, and the like, data may be archived, e.g., historical data, such that the operator may recall the data to determine the quality of the compression or decompression and to provide feedback to the operator in order to improve his or her compression and/or decompression technique(s). More particularly, all of the statistical, quantitative, qualitative, and the like, historical or archived data utilized in generating the elasticity image, and each reference data frame used in composing the elasticity image, may be displayed in a statistical, quantitative, qualitative, and the like, diagram such as a table, chart, graph and the like, as known to one skilled in the art, with or without the elasticity image. For the purpose of example, and not to be limiting, such a diagram may comprise the graphs and charts of FIGS. 6-9, each alone or in combination with each other and/or the resultant elasticity image or pertinent reference data frame, arranged on a display unit for the operator, supervisor and the like.

The operator and/or supervisor may also receive feedback utilizing more than a diagram. For example, these diagrams may also include color and/or grayscale images of compression motions and/or decompression motions. An operator may determine the quality of a compression and/or a decompression by viewing a color change, or one or more color changes, occurring during a compression motion, e.g., the brightening of a darker area to a lighter area in a grayscale or color image, or the change in color from grayscale to color, and the like. A diagram exhibiting such color images and/or color changes may also be archived, e.g., historical data, and recalled during and/or after generating an elasticity image.

In addition to displaying archived or historical data using diagrams, audible noises may also be employed, and archived, to provide feedback to the operator. An audio recording and playback device may be integrated within elasticity imaging system 10, or may stand alone and be capable of capturing the audible noises produced while performing elasticity imaging. A noise may translate to a compression motion, a decompression motion, an acceptable compression/decompression motion, an unsatisfactory compression/decompression motion, and the like. Such noises may communicate information using one or more pitches, harmonics, volumes, rhythms, beats, combinations comprising at least one of the foregoing, and the like. The operator may hear such noises while compressing and decompressing a biological tissue and learn whether or not the motions fall within an acceptable compression/decompression range. Likewise, a supervisor may recall and listen to the recorded noise patterns to determine the quality of the compressions/decompressions performed by the operator. In turn, an operator may continue learning how to improve his/her skills by listening to an audio recording of his/her experimental runs using an elasticity imaging system contemplated herein.

It is to be understood that the invention is not limited to the illustrations described and shown herein, which are deemed to be merely illustrative of the best modes of carrying out the invention, and which are susceptible of modification of form, size, arrangement of parts and details of operation. The invention rather is intended to encompass all such modifications which are within its spirit and scope as defined by the claims.

What is claimed is:

1. A process for performing elasticity imaging on a biological tissue, comprising:
selecting automatically based upon at least one criterion at least one frame pair comprising a pre-compression frame and a post-compression frame;
analyzing said at least one frame pair; calculating an elasticity image; and
displaying said elasticity image.

2. The process of claim 1, wherein the automatic selection step comprises using a compression feedback algorithm.

3. The process of claim 1, wherein said at least one criterion comprises an amount of tissue displacement and at least one tissue correlation result.

4. The process of claim 1, wherein the automatic selection step further comprises predicting an elasticity image quality prior to calculating an elasticity image.

5. The process of claim 1, wherein the automatic selection step further comprises providing to an operator at least one of the following: a visual feedback or an audible feedback or both said visual feedback and said audible feedback.

6. The process of claim 5, wherein the providing step further comprises providing said visual feedback and said audible feedback to said operator upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion.

7. The process of claim 1, further comprising confirming off-line the quality of a plurality of data used in the calculation of said elasticity image.

8. The process of claim 7, wherein the confirmation step comprises displaying visually and projecting audibly at least one of the following: at least one quantitative data, at least one qualitative data, or both said at least one quantitative data and said at least one qualitative data.

9. The process of claim 7, wherein the confirmation step comprises displaying visually or projecting audibly at least one of the following: at least one quantitative data, at least one qualitative data, or both said at least one quantitative data and said at least one qualitative data.

10. A process for performing elasticity imaging, comprising:
setting a region of interest about an image;
deforming a biological tissue to create a tissue deformation;
acquiring at least two RF frame data at an imaging-relevant frame rate;
introducing said at least two RF frame data into a compression feedback algorithm;
determining at least one quantitative indication of a tissue deformation quality for said at least two RF frame data within at least one block from said region of interest using a block matching algorithm;
comparing said at least one quantitative indication of said at least two RF frame data to at least one of a plurality of threshold values within at least one block from said region of interest;
displaying said comparison of said at least one quantitative indication of said at least two RF frame data to at least one of said plurality of threshold values;
predicting an acceptable tissue deformation based upon said comparison;
determining said predicted acceptable tissue deformation is satisfactory to yield a satisfactory tissue deformation; and
displaying an elasticity image of said biological tissue.

11. The process of claim 10, wherein the determination step of said quantitative indication comprises calculating at least one axial compression magnitude value and at least one lateral compression magnitude value.

12. The process of claim 11, wherein the calculation step comprises the steps of:
estimating at least one axial shift and at least one lateral shift between at least two adjacent RF data frames within at least one block from said region of interest;
cumulating said at least one axial shift and said at least one lateral shift to generate said axial compression magnitude value and said lateral compression magnitude value between a reference RF frame data and a current RF frame data within said at least one block from said region of interest.

13. The process of claim 12, wherein the estimation process of the at least one axial shift and at least one lateral shift comprises executing a search procedure over at least one axial search range and at least one lateral search range within at least one block from said region of interest.

14. The process of claim 13, further comprising displaying at least one graphical representation of at least one direction of said tissue deformation and a magnitude of said tissue deformation based upon said axial compression magnitude value and said lateral compression magnitude value.

15. The process of claim 13, wherein said reference RF frame data and said current RF frame data are not adjacent.

16. The process of claim 13, wherein the estimation step comprises estimating said at least one axial shift and said at least one lateral shift using said block matching algorithm.

17. The process of claim 16, wherein said block matching algorithm comprises a correlation coefficient technique.

18. The process of claim 17, wherein the correlation coefficient technique further comprises the steps of:
applying an envelope function to a set of correlation coefficients obtained during said search procedure over at least one axial search range and at least one lateral search range to generate a set of envelope coefficients;
identifying a maximum value of said set of envelope coefficients;
determining an axial lag of said maximum value indicating an axial displacement; and
determining a lateral lag of said maximum value indicating indicating a lateral displacement.

19. The process of claim 10, wherein the determination of said at least one quantitative indication of said tissue deformation quality step further comprises calculating at least one compression score within at least one block from said region of interest between a first envelope of a reference RF frame data and a second envelope of a current RF frame data.

20. The process of claim 19, wherein the calculation step comprises calculating said at least one compression score using a normalized correlation technique.

21. The process of claim 20, wherein said normalized correlation technique is a correlation coefficient technique.

22. The process of claim 19, wherein the calculation step comprises using an axial compression magnitude and a lateral compression magnitude to compensate for a motion between a reference RF frame data and a current RF frame data.

23. The process of claim 10, wherein the comparison step comprises the steps of:
comparing at least one compression score with a least acceptable compression score threshold value;
comparing an absolute value of a lateral compression magnitude with a greatest acceptable lateral threshold value;
comparing an axial compression magnitude value with a greatest acceptable axial threshold value and an imaging acceptable threshold value; and
comparing said axial compression magnitude value with a zero value.

24. The process of claim 10, wherein the display step of said comparison further comprises displaying said at least one quantitative indication and said at least one of said plurality of threshold values.

25. The process of claim 24, wherein the display step further comprises displaying a quantitative axial displacement for said at least one block from said region of interest.

26. The process of claim 25, wherein the display step comprises displaying a graphical representation of said quantitative axial displacement.

27. The process of claim 26, wherein the display step comprises displaying a color coded graphical representation.

28. The process of claim 24, wherein the display step further comprises displaying a greatest acceptable axial threshold value and an imaging acceptable threshold value of said at least one quantitative indication.

29. The process of claim 28, wherein the display step comprises displaying a color coded graphical representation.

30. The process of claim 24, wherein the display step further comprises displaying a greatest acceptable axial threshold value as a maximum value or a minimum value in a graphical representation.

31. The process of claim 24, wherein the display step further comprises displaying an imaging acceptable threshold value as a maximum value or a minimum value in a graphical representation.

32. The process of claim 10, wherein the display step of said comparison further comprises the steps of:
displaying a quantitative representation of at least one cumulated lateral displacement value or at least one cumulated axial displacement value for said at least one block from said region of interest.

33. The process of claim 32, wherein the display step comprises displaying a color coded quantitative representation.

34. The process of claim 10, wherein the display step of said comparison further comprises displaying an absolute value of at least one cumulated lateral displacement or at least one cumulated axial displacement value for said at least one block.

35. The process of claim 10, wherein the display step of said comparison further comprises displaying a greatest acceptable lateral threshold value as a maximum value or a minimum value in a graphical representation.

36. The process of claim 10, wherein the display step of said comparison further comprises displaying a quantitative representation of a compression score for said at least one block.

37. The process of claim 36, wherein the display step comprises displaying a color coded graphical representation.

38. The process of claim 36, wherein the display step comprises displaying a least acceptable compression score threshold value.

39. The process of claim 36, wherein the display step comprises displaying a least acceptable compression score threshold value as a maximum value or a minimum value in a graphical representation.

40. The process of claim 10, wherein the determination step of said predicted acceptable tissue deformation for said at least one block from said region of interest comprises the steps of:
determining a compression score value is greater than a least acceptable compression score threshold value;
determining an absolute value of a lateral compression magnitude value is less than a greatest acceptable lateral threshold value; and
determining an axial compression magnitude value is a positive value and less than a greatest acceptable axial threshold value.

41. The process of claim 40, further comprising determining an unacceptable tissue deformation in the event of any one of the following:
determining said compression score value is less than said least acceptable compression score threshold value;
determining said absolute value of said lateral compression magnitude value is greater than said greatest acceptable lateral threshold value; and
determining said axial compression magnitude value is a negative value or a positive value greater than said greatest acceptable axial threshold value.

42. The process of claim 41, further comprising the steps of:
copying said RF frame data from an RF current frame buffer to an RF reference frame buffer;
copying said RF frame data from said RF current frame buffer to an RF previous frame buffer;
setting said axial compression magnitude value and said lateral compression magnitude value to zero;
restarting said compression feedback algorithm;
acquiring at least one RF frame data; and
storing said at least one RF frame data in said RF current frame buffer.

43. The process of claim 10, wherein the determination step of said satisfactory tissue deformation for said at least one block from said region of interest comprises determining an axial compression magnitude value is greater than an imaging acceptable threshold value.

44. The process of claim 43, further comprising determining an unsatisfactory acceptable tissue deformation when said axial compression magnitude value is less than said imaging acceptable threshold value.

45. The process of claim 44, further comprising the steps of:
copying said RF frame data from said RF current frame buffer to an RF previous frame buffer;
restarting said compression feedback algorithm;
acquiring at least one RF frame data; and
storing said at least one RF frame data in said RF current frame buffer.

46. The process of claim 10, wherein after the display of said elasticity image step further comprising the steps of:
copying said RF frame data from an RF current frame buffer to an RF reference frame buffer;
copying said RF frame data from an RF current frame buffer to an RF previous frame buffer;
setting an axial compression magnitude value and a lateral compression magnitude value to zero;
restarting said compression feedback algorithm;
acquiring at least one RF frame data; and
storing said at least one RF frame data in said RF current frame buffer.

47. The process of claim 10, further comprising archiving said at least two RF frame data, said elasticity image, a plurality of data of a reference axial displacement buffer, a plurality of data of a reference lateral displacement buffer and a plurality of data of a compression score buffer.

48. The process of claim 47, wherein the archiving step further comprises providing access to review said at least two RF frame data, said elasticity image, said plurality of data of said reference axial displacement buffer, said plurality of data of said reference lateral displacement buffer and said plurality of data of said compression score buffer.

49. The process of claim 48, wherein the providing access step further comprises providing access off-line for training an operator, assessing elasticity image quality and confirming elasticity image quality.

50. The process of claim 10, further comprising generating at least one audible noise upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion.

51. The process of claim 50, wherein each of said at least one audible noise corresponding to any one of the following: said compression motion, said decompression motion, said acceptable compression motion, said acceptable decompression motion, said unacceptable compression motion, said unacceptable decompression motion, said satisfactory compression motion, said satisfactory decompression motion, said unsatisfactory compression motion, or said unsatisfactory decompression motion.

52. The process of claim 50, further comprising recording said at least one audible noise.

53. The process of claim 52, further comprising amplifying the recorded said at least one audible noise.

54. The process of claim 10, further comprising generating and displaying at least one colored image corresponding to any one of the following: a compression motion, a decompression motion, an acceptable compression motion, an acceptable decompression motion, an unacceptable compression motion, an unacceptable decompression motion, an unsatisfactory compression motion, or an unsatisfactory decompression motion.

55. The process of claim 54, further comprising comparing a first colored image to a second colored image.

56. The process of claim 55, further comprising detecting a change in at least one color upon comparison of said first colored image to said second colored image.

57. The process of claim 56, wherein the detection step comprises detecting a change in an intensity of said at least one color.

58. The process of claim 56, wherein the detection step comprises detecting a change in a shade of said at least one color.

59. The process of claim 10, further comprising the additional step of generating an elasticity image of said biological tissue based upon achieving an acceptable compression prior to displaying said elasticity image.

60. An ultrasound system comprising a computer readable storage device readable by the system, tangibly embodying a program having a set of instructions executable by the system to perform the following steps for performing elasticity imaging, the set of instructions comprising:
an instruction to set a region of interest about an image followed by the deformation of a biological tissue to create a tissue deformation;
an instruction to acquire at least two RF frame data at an imaging-relevant frame rate;
an instruction to introduce said at least two RF frame data into a compression feedback algorithm;
an instruction to determine at least one quantitative indication of a tissue deformation quality for said at least two RF frame data within at least one block from said region of interest using a block matching algorithm;
an instruction to compare said at least one quantitative indication of said at least two RF frame data to at least one of a plurality of threshold values within at least one block from said region of interest;
an instruction to display said comparison of said at least one quantitative indication of said at least two RF frame data to at least one of said plurality of threshold values;
an instruction to predict an acceptable tissue deformation based upon said comparison;
an instruction to determine said predicted acceptable tissue deformation is satisfactory to yield a satisfactory tissue deformation; and
an instruction to display an elasticity image of said biological tissue.

61. The ultrasound system of claim 60, wherein the determination instruction of said quantitative indication comprises an instruction to calculate at least one axial compression magnitude value and at least one lateral compression magnitude value.

62. The ultrasound system of claim 61, wherein the calculation instruction comprises:
an instruction to estimate at least one axial shift and at least one lateral shift between at least two adjacent RF data frames within at least one block from said region of interest; and
an instruction to cumulate said at least one axial shift and said at least one lateral shift to generate said axial compression magnitude value and said lateral compression magnitude value between a reference RF frame data and a current RF frame data within at least one block from said region of interest.

63. The ultrasound system of claim 62, wherein the estimation instruction of said at least one axial shift and said at least one lateral shift comprises executing a search procedure over at least one axial search range and at least one lateral search range within at least one block from said region of interest.

64. The ultrasound system of claim 63, further comprising an instruction to display at least one graphical representation of at least one direction of said tissue deformation and a magnitude of said tissue deformation based upon said axial compression magnitude value and said lateral compression magnitude value.

65. The ultrasound system of claim 63, wherein said reference RF frame data and said current RF frame data are not adjacent.

66. The ultrasound system of claim 63, wherein the estimation instruction comprises an instruction to estimate said at least one axial shift and said at least one lateral shift using said block matching algorithm.

67. The ultrasound system of claim 66, wherein said block matching algorithm comprises a correlation coefficient technique.

68. The ultrasound system of claim 67, further comprising an instruction to apply the correlation coefficient technique:
an instruction to apply an envelope function to a set of correlation coefficients obtained during said search procedure over at least one axial search range and at least one lateral search range to generate a set of envelope coefficients;
an instruction to identify a maximum value of said set of envelope coefficients;
an instruction to determine an axial lag of said maximum value indicating an axial displacement; and
an instruction to determine a lateral lag of said maximum value indicating indicating a lateral displacement.

69. The ultrasound system of claim 66, wherein the determination instruction of said at least one quantitative indication of said tissue deformation further comprises an instruction to calculate at least one compression score within at least one block from said region of interest between a first envelope of a reference RF frame data and a second envelope of a current RF frame data.

70. The ultrasound system of claim 69, wherein the calculation instruction comprises an instruction to calculate said at least one compression score using a normalized correlation technique.

71. The ultrasound system of claim 70, wherein said normalized correlation technique is a correlation coefficient technique.

72. The ultrasound system of claim 69, wherein the calculation instruction comprises an instruction to use an axial compression magnitude and a lateral compression magnitude to compensate for a motion between a reference RF frame data and a current RF frame data.

73. The ultrasound system of claim 60, wherein the comparison instruction comprises:
an instruction to compare at least one compression score with a least acceptable compression score threshold value;
an instruction to compare an absolute value of a lateral compression magnitude with a greatest acceptable lateral threshold value;
an instruction to compare an axial compression magnitude value with a greatest acceptable axial threshold value and an imaging acceptable threshold value; and
an instruction to compare said axial compression magnitude value with a zero value.

74. The ultrasound system of claim 60, wherein the display instruction of said comparison further comprises an instruction to display said at least one quantitative indication and said at least one of said plurality of threshold values.

75. The ultrasound system of claim 74, wherein the display instruction further comprises an instruction to display a quantitative axial displacement for said at least one block from said region of interest.

76. The ultrasound system of claim 75, wherein the display instruction comprises an instruction to display a graphical representation of said quantitative axial displacement.

77. The ultrasound system of claim 76, wherein the display instruction comprises an instruction to display a color coded graphical representation.

78. The ultrasound system of claim 74, wherein the display instruction further comprises an instruction to display a greatest acceptable axial threshold value and an imaging acceptable threshold value of said at least one quantitative indication.

79. The ultrasound system of claim 78, wherein the display instruction comprises an instruction to display a color coded graphical representation.

80. The ultrasound system of claim 74, wherein the display instruction further comprises an instruction to display a greatest acceptable axial threshold value as a maximum value or a minimum value in a graphical representation.

81. The ultrasound system of claim 74, wherein the display instruction further comprises an instruction to display an imaging acceptable threshold value as a maximum value or a minimum value in a graphical representation.

82. The ultrasound system of claim 60, wherein the display instruction of said comparison further comprises:
an instruction to display a quantitative representation of at least one cumulated lateral displacement value or at least one cumulated axial displacement value for said at least one block from said region of interest.

83. The ultrasound system of claim 82, wherein the display instruction comprises an instruction to display a color coded quantitative representation.

84. The ultrasound system of claim 60, wherein the display instruction of said comparison further comprises an instruction to display an absolute value of at least one cumulated lateral displacement or at least one cumulated axial displacement value for said at least one block.

85. The ultrasound system of claim 60, wherein the display instruction of said comparison further comprises an instruction to display a greatest acceptable lateral threshold value as a maximum value or a minimum value in a graphical representation.

86. The ultrasound system of claim 60, wherein the display instruction of said comparison further comprises an instruction to display a quantitative representation of a compression score for said at least one block.

87. The ultrasound system of claim 86, wherein the display instruction comprises an instruction to display a color coded graphical representation.

88. The ultrasound system of claim 86, wherein the display instruction comprises an instruction to display a least acceptable compression score threshold value.

89. The ultrasound system of claim 86, wherein the display instruction comprises an instruction to display a least acceptable compression score threshold value as a maximum value or a minimum value in a graphical representation.

90. The ultrasound system of claim 60, wherein the determination instruction of said predicted acceptable tissue deformation for said at least one block from said region of interest comprises:
an instruction to determine a compression score value is greater than a least acceptable compression score threshold value;
an instruction to determine an absolute value of a lateral compression magnitude value is less than a greatest acceptable lateral threshold value; and
an instruction to determine an axial compression magnitude value is a positive value and less than a greatest acceptable axial threshold value.

91. The ultrasound system of claim 90, further comprising an instruction to determine an unacceptable tissue deformation in the event of any one of the following: said compression score value is less than said least acceptable compression score threshold value, or said absolute value of said lateral compression magnitude value is greater than said greatest acceptable lateral threshold value, or said axial compression magnitude value is a positive value and greater than said greatest acceptable axial threshold value.

92. The ultrasound system of claim 91, further comprising:
an instruction to copy said RF frame data from an RF current frame buffer to an RF reference frame buffer;
an instruction to copy said RF frame data from said RF current frame buffer to an RF previous frame buffer;
an instruction to set said axial compression magnitude value and said lateral compression magnitude value to zero;
an instruction to restart said compression feedback algorithm;
an instruction to acquire at least one RF frame data; and
an instruction to store said at least one RF frame data in said RF current frame buffer.

93. The ultrasound system of claim 60, wherein the determination instruction of said satisfactory tissue deformation for said at least one block from said region of interest comprises an instruction to determine an axial compression magnitude value is greater than an imaging acceptable threshold value.

94. The ultrasound system of claim 93, further comprising an instruction to determine an unsatisfactory acceptable tissue deformation when said axial compression magnitude value is less than said imaging acceptable threshold value.

95. The ultrasound system of claim 94, further comprising:
an instruction to copy said RF frame data from said RF current frame buffer to an RF previous frame buffer;
an instruction to restart said compression feedback algorithm;
an instruction to acquire at least one RF frame data; and
an instruction to store said at least one RF frame data in said RF current frame buffer.

96. The ultrasound system of claim 60, wherein after the display of said elasticity image instruction, further comprising:
an instruction to copy said RF frame data from an RF current frame buffer to an RF reference frame buffer;
an instruction to copy said RF frame data from an RF current frame buffer to an RF previous frame buffer;
an instruction to set an axial compression magnitude value and a lateral compression magnitude value to zero;
an instruction to restart said compression feedback algorithm;
an instruction to acquire at least one RF frame data; and
an instruction to store said at least one RF frame data in said RF current frame buffer.

97. The ultrasound system of claim 60, further comprising an instruction to archive said at least two RF frame data, said elasticity image, a plurality of data of a reference axial displacement buffer, a plurality of data of a reference lateral displacement buffer and a plurality of data of a compression score buffer.

98. The ultrasound system of claim 97, wherein the archiving instruction further comprises an instruction to provide access to review said at least two RF frame data, said elasticity image, said plurality of data of said reference axial displacement buffer, said plurality of data of said reference lateral displacement buffer and said plurality of data of said compression score buffer.

99. The ultrasound system of claim 98, wherein the providing access instruction further comprises an instruction to provide access off-line for training an operator, assessing elasticity image quality and confirming elasticity image quality.

100. The device of claim 60, further comprising an instruction to generate at least one audible noise upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion.

101. The ultrasound system of claim 100, wherein each of said at least one audible noise correspond to any one of the following: said compression motion, said decompression motion, said acceptable compression motion, said acceptable decompression motion, said unacceptable compression motion, said unacceptable decompression motion, said satisfactory compression motion, said satisfactory decompression motion, said unsatisfactory compression motion, or said unsatisfactory decompression motion.

102. The ultrasound system of claim 100, further comprising an instruction to record said at least one audible noise.

103. The ultrasound system of claim 102, further comprising an instruction to amplify the recorded said at least one audible noise.

104. The ultrasound system of claim 60, further comprising an instruction to generate and display at least one colored image corresponding to any one of the following: a compression motion, a decompression motion, an acceptable compression motion, an acceptable decompression motion, an unacceptable compression motion, an unacceptable decompression motion, an unsatisfactory compression motion, or an unsatisfactory decompression motion.

105. The ultrasound system of claim 104, further comprising an instruction to compare a first colored image to a second colored image.

106. The ultrasound system of claim 105, further comprising an instruction to detect a change in at least one color upon comparison of said first colored image to said second colored image.

107. The ultrasound system of claim 106, wherein the detection instruction comprises an instruction to detect a change in an intensity of said at least one color.

108. The ultrasound system of claim 106, wherein the detection instruction comprises an instruction to detect a change in a shade of said at least one color.

109. The ultrasound system of claim 60, further comprising an instruction to generate an elasticity image of said biological tissue based upon achieving an acceptable compression prior to displaying said elasticity image.

110. An ultrasound system comprising a computer readable storage device readable by the system, tangibly embodying a program having a set of instructions executable by the system to perform the following steps for performing elasticity imaging, the set of instructions comprising:
an instruction to select automatically based upon at least one criterion at least one frame pair comprising a pre-compression frame and a post-compression frame;
an instruction to analyze said at least one frame pair;
an instruction to calculate an elasticity image; and
an instruction to display said elasticity image.

111. The ultrasound system of claim 110, wherein the automatic selection instruction comprises an instruction to use a compression feedback algorithm.

112. The ultrasound system of claim 110, wherein said at least one criterion comprises an amount of tissue displacement and at least one tissue correlation result.

113. The ultrasound system of claim 110, wherein the automatic selection instruction further comprises an instruction to predict an elasticity image quality prior to calculating an elasticity image.

114. The ultrasound system of claim 110, wherein the automatic selection instruction further comprises an instruction to provide to an operator at least one of the following: a visual feedback or an audible feedback or both said visual feedback and said audible feedback.

115. The ultrasound system of claim 114, wherein the providing instruction further comprises an instruction to provide said visual feedback and said audible feedback to said operator upon achieving any one of the following: a compression motion, a decompression motion, an acceptable compression motion, an acceptable decompression motion, an unacceptable compression motion, an unacceptable decompression motion, a satisfactory compression motion, a satisfactory decompression motion, an unsatisfactory compression motion, or an unsatisfactory decompression motion.

116. The ultrasound system of claim 114, further comprising an instruction to confirm off-line the quality of a plurality of data used in the calculation of said elasticity image.

117. The ultrasound system of claim 116, wherein the confirmation instruction comprises an instruction to display visually and project audibly at least one of the following: at least one quantitative data, at least one qualitative data, or both said at least one quantitative data and said at least one qualitative data.

118. The ultrasound system of claim 116, wherein the confirmation instruction comprises an instruction to display visually or project audibly at least one of the following: at least one quantitative data, at least one qualitative data, or both said at least one quantitative data and said at least one qualitative data.
